(19) United States
(12) Patent Application Publication
(10) Pub. No.: US 2020/0022652 A1
(43) Pub. Date: Jan. 23, 2020

(54) A PROBE

(71) Applicant: Osstell AB, Gatebarg (SE)

(72) Inventor: Neil MEREDITH, Queensland (AU)

(21) Appl. No.: 16/337,960

(22) PCT Filed: Sep. 28, 2017
(86) PCT No.: PCT/IB2017/055969

§ 371 Date: Mar. 29, 2019

(30) Foreign Application Priority Data
Sep. 29, 2016 (GB) 1616548.2

(51) Int. Cl. A61B 5/00; A61C 19/04

(52) U.S. Cl. CPC A61B 5/6878; A61B 5/0051; A61B 5/4542; A61C 19/04; A61B 5/742; A61B 5/7225; A61B 5/4509; A61B 5/7275

(57) ABSTRACT

A method of assessing the stiffness of bone comprising the steps of: placing an elongate probe into a predrilled aperture in the bone to be assessed; exciting the elongate probe to physically oscillate; and monitoring the resonance frequency of the probe. The resonance frequency is analysed to determine the quality, density and/or stiffness of the bone.

### FIELD OF THE INVENTION

The invention relates to a method of measuring bone quality, density and/or stiffness, particularly in relation to jaws and dental implants,

### BACKGROUND TO THE INVENTION

Frequently, dental implants are in the form of metal screws that are inserted into the jaw bone as a means of anchoring crowns, bridges or dentures. Such implants are normally threaded, often made of titanium, zirconia or their alloys, and are inserted into a hole that is prepared in the bone prior to insertion of the implant. The implants come in a wide range of sizes, typically 3-10 mm diameter and 5-20 mm in length.

The fixing and stability of an implant at insertion is critical to its success as bone grows on to the implant surface and this process is disturbed if the stability is low, the fixity is poor or if there is motion of the implant in the bone during the healing process.

A technique exists to measure the stability of an implant in bone by measuring the resonance frequency of an attachable member (Meredith and Cawley). This is intended to measure the stability of an implant at placement, comparing it with reference measurements, and also to measure the changes in stability during the healing process after placement. However, this process only gives information about an implant that is in situ in the bone.

Bone quality has been classified subjectively (Lekholm and Zarb) in terms of quality (1−4 4=bad) and quantity (A−E E=bad). Obviously some determination of quantity can be made prior to surgery from x-ray radiographs and at the time of surgery by visual examination, However, it is very difficult to obtain quantitative information about quality at any time.

Bone is a complex heterogeneous structural material having anisotropic and sometimes orthotropic behaviour. Characteristically, it has two broad types; cortical and trabecular bone, as shown in FIG. 1. Cortical bone is often the outer layer of the bone and it is dense and has relatively few blood vessels. The thickness of this layer varies and typically in the upper lower jaw might be between 1 mm and 10 mm. The Trabecular, inner part of the bone is much softer and often contains the marrow spaces and blood supply. It occupies the internal space inside the cortex. The Trabecular bone may not exist at all or could be up to 20 mm in the facial bones.

The amount, quantity and relative ratio of these two types of bone will determine the stability of an inserted implant, particularly a dental implant. The stiffness of cortical bone and its Elastic Modulus is typically ten times great than that of trabecular bone. Therefore, it is obvious that Cortical bone is critical to the stability of an implant. It is also important that an implant is placed inside the bone as there is risk of injury if an implant perforates the bone at an exit point, thereby risking damaging anatomical structures including nerves, blood vessels and sinuses.

Currently, there is no satisfactory method to measure and predict bone quality prior to implant placement. Examination of x-ray radiographs gives a qualitative indication of bone quantity and quality and bone density can be measured quantitatively from radiographs on the Hounsfield Scale that is used in typing tumours. However, this single value is impossible to interpret as a measure of potential implant stability.

One method that has been used with limited success is the measurement of insertion torque or the thread cutting force used during the insertion of an implant. This is typically performed by measuring the back EMF or current drawn by the motorized hand-piece used to place the implant. The data is typically presented as a graph. This method has a number of significant disadvantages; for example, a measurement is only made when the implant is inserted so there is no information available to assist in the selection of the implant type, geometry or size. An additional potential problem is that there are many interrelated factors influencing the insertion torque measurement, which can include: the size and shape of the implant; the width of the implant relative to the width of the prepared hole; geometrical features such as flanges that may halt progression the implant; and friction created by bone chips from cutting and other factors. These all interrelate making a true measurement of bone quality impossible under such a method.

### SUMMARY OF THE INVENTION

Accordingly, the present invention is directed to a method of assessing the stiffness of bone comprising the steps of:
placing an elongate probe into a predrilled aperture in the bone to be assessed;
exciting the elongate probe to physically oscillate; and
monitoring the resonance frequency of the probe;
wherein the resonance frequency is analysed to determine the quality, density and/or stiffness of the bone.

In the present invention an aperture, or hole, is drilled into the bone using a drill bit, and a probe is inserted into the hole prior to any implant being inserted. This allows a practitioner to have knowledge of the stiffness and quality of the bone prior to placement of an implant. As a result, where the characteristics of the bone can be determined accurately, then the most appropriate design, geometry and size of implant can be chosen from the many thousands of types available on the market to increase the stability and resistance to loading. This allows for more predictable and reliable insertion and retention of implants.

The present invention employs a diagnostic probe that is vibrated electromechanically. Thus, when the probe is inserted into a predrilled hole in bone, the vibration is damped mechanically by the surrounding bone and this can be measured as a decrease in amplitude or alternatively a shift in the resonance frequency of the member. It is possible to control external variables, thereby giving a truly quantitative measurement of bone quality and stiffness for the aperture, which is to become the implant site.

In the present invention, the probe behaves as a cantilever beam. The resonance frequency of such a cantilever beam is a function of its modulus, length and cross sectional area. Thus the resonance frequency of the probe will be determined by the depth to which it is inserted in the hole and the stiffness of the inserted portion.

Preferably, the method comprises the further step of removing the probe from the bone once the analysis has been undertaken. Once the bone has been analysed, the probe is removed from the aperture in the bone to allow for the required implant to be inserted. The probe is intended to be employed as a measurement probe and it does not form part of, nor is it attached to, any implant components that are intended to be inserted in the body, either permanently or temporarily. Whilst it is envisaged that the probe could be used as the implant in some circumstances, it is preferable that the probe is removed to allow the more appropriate implant to be employed.

In one arrangement, the resultant output signal from the probe is amplified and/or filtered before being analysed. Advantageously, the analysis is undertaken by a central processing unit and wherein the central processing unit further comprises non-volatile memory, and, in one arrangement, a look-up table and/or calibration data is provided on the non-volatile memory and the information thereupon is accessed by the central processing unit during analysis of the resonance frequency. By readily providing calibration information and/or a look-up table the information can be readily interpreted and processed to provide a meaningful result.

Once the analysis of the bone has been undertaken, it may be that a graphical display of the calculated quality, density and/or stiffness is produced. This enables the operator to more clearly understand the characteristics of the bone structure.

In a preferable arrangement, the probe is provided with markings along its length to indicate the depth to which it is inserted into the aperture. By having an indication of the depth to which the probe is inserted, the length of the implant can be accurately measured prior to its insertion into the aperture. This allows for the implant to be sized correctly before it needs to be inserted. The probe may be repeatably inserted into the pilot hole to any depth up to the depth drilled by the pilot drill.

It may be advantageous that the probe is provided with a threaded or spiral external profile, and it may be further advantageous that the size and profile of the probe is matched to that of the drill bit used to create the aperture. The probe should be designed to have a constant stiffness between it and the surround bone to be measured. This may be achieved by matching the diameter and profile of the pilot drill with that of the probe. Additionally, or alternatively, the probe may be smooth along its length or have a spiral or threaded profile enabling insertion by pushing, rotation or a combination of the two.

The invention extends to apparatus for putting the present invention into effect and a probe therefor.

### BRIEF DESCRIPTION OF THE DRAWINGS

Embodiments of the invention will now be described, by way of example only, and with reference to the accompanying drawings, in which:

FIG. 2 shows probes for use in the method of the present invention;

FIG. 3 is a diagram showing a schematic of an arrangement for use in the present invention; and

FIG. 4 chart showing the relationship between resonance frequency and bone quality.

### DESCRIPTION OF EXEMPLARY EMBODIMENT

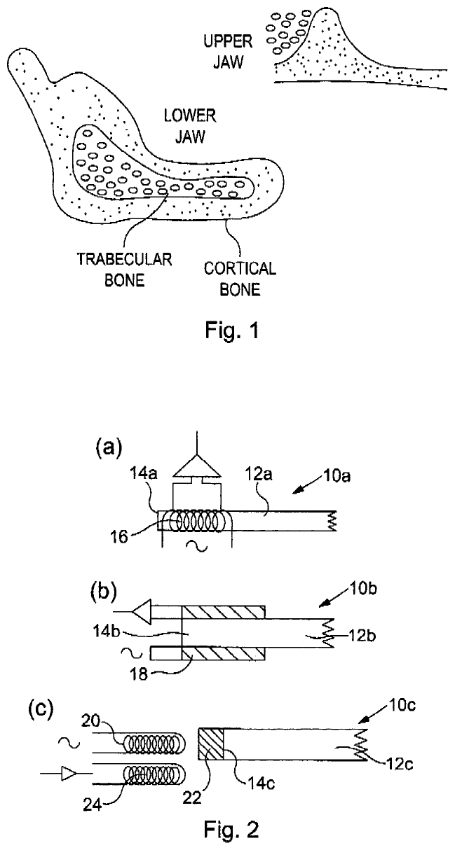

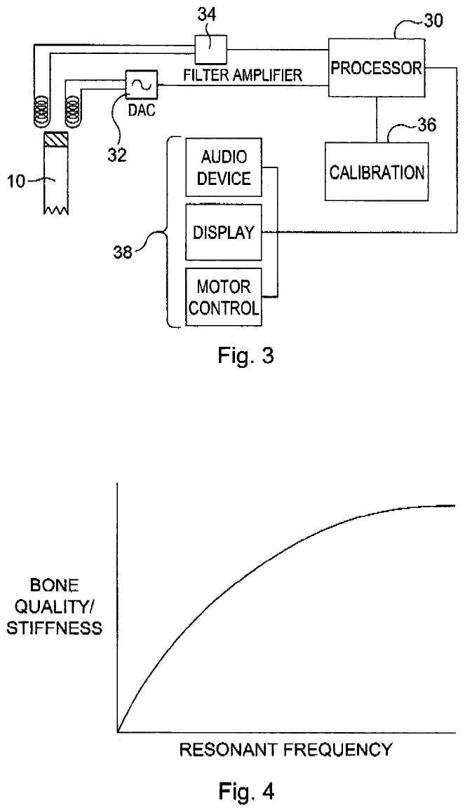

FIG. 2 shows probes 10 comprising an elongate section 12 and a top section 14. The top section is provided with activation means for exciting the probe 10, which include:
a) a direct electromagnetic connection comprising coils 16 wrapped around or bonded to the top section 16, in which the coils cause excitation and also measure the response of the probe.
b) a direct piezoelectric connection 18 comprising piezoelectric crystals that are attached to, and may be bonded to, the probe 10 to act as transmitters and receivers.
c) an indirect electromagnetic connection 20 comprising a magnet or ferrous material 22 at the top section 14c , thereby allowing part or all of the probe to be excited and measured by remote coils 24 in close proximity of the probe 10 to create an inductive effect.

The probe 10 has two ends: a first, passive end with no attachments; and a second end 14 that is provided with connection means 16, 18 and 20 to electromechanically excite the probe 10 and to measure the resulting resonance frequencies.

The apparatus comprises the probe 10 and excitation means connected directly or indirectly thereto. A central processing unit 30 is provided that sends an alternating current signal to the excitation means. The amplitude, waveform and character of this signal will match the appropriate requirements of the specific transducer used in the excitation means. This signal is synthesized digitally by the central processing unit 30 and passed through a digital to analogue converter 32.

The resultant output signal from the probe 10 is be amplified, filtered and conditioned by a filter-amplified 34 and then analysed by the central processing unit 30. The central processing unit 30 obtains information from a look-up table to compare measured and calibration data 36 to generate a quantitative value or graphical display, which is provided by an output 38 for interpretation by the operator.

During implant site preparation a number of drill bits of increasing diameter and/or length are typically used. This serves a number of purposes, including changing the potential alignment during preparation, reducing heat generation by cutting in small stages and shaping the drill hole for the implant size and shape. As such, it is very common to use a small, typically a 2 mm diameter drill, to prepare a pilot hole in the bone, however, the drill could range from 1 mm to 10 mm depending upon the requirements. Once the hole has been created, a probe can be inserted at least partially into the hole.

The characteristics of the probe, for example its shape and length, will depend upon the requirements. Therefore, whilst the cross section of the probe is, preferentially, circular, it may be oval, square or irregular, and it may have geometrical features. The diameter of the probe may vary in the range of 1 mm to 10 mm, although around 2 mm is the preferred diameter.

It is important that the probe is designed to have a constant stiffness between it and the bone it is measuring in order to achieve a reliable measurement. This is addressed by matching the diameter and profile of the pilot drill with that of the probe in order to enables the interfacial stiffness to be eliminated in any measurement of bone quality.

The probe can be readily calibrated by test holes in samples of homogeneous, isotropic materials that simulate bones' mechanical properties. This data enables the probe to be calibrated to give a value of bone quality and quantity as a function of resonance frequency. This technique can also be applied to damping measurements from the probe.

The probe may comprise metal, typically aluminium or titanium, and/or other materials. Ideally, the probe comprises material(s) that are intended to resist corrosion in the surgical environment and during sterilization.

A wide range of alignments and orientations exist for the transducers attached to the probe and these may be used to gather further data related to orientation.

It will be appreciated that an excited cantilever beam may exhibit a number of resonance frequencies related to its modes of vibration. The probe may measure any or all of these modes if present.

The present invention may be employed to measure bone quality in diseases where bone density or quality may be reduced, for example in osteoporosis, osteomalacia or vitamin deficiencies. Additionally, it may be employed not just in dental situations but also in respect of orthopaedic matters where it is advantageous to know the bone quality and stiffness.

1. A method of assessing the quality, density and/or stiffness of bone comprising the steps of:
placing an elongate probe into a predrilled aperture in the bone to be assessed;
exciting the elongate probe to physically oscillate; and
monitoring the resonance frequency of the probe;
wherein the resonance frequency is analysed to determine the quality, density and/or stiffness of the bone.

2. A method according to claim 1, wherein the method comprises the further step of removing the probe from the bone once the analysis has been undertaken.

3. A method according to claim 1, wherein the resultant output resonance frequency is amplified and/or filtered before being analysed.

4. A method according to claim 1, wherein the analysis is undertaken by a central processing unit and wherein the central processing unit further comprises non-volatile memory.

5. A method according to claim 4, wherein a look-up table and/or calibration data is provided on the non-volatile memory and the information thereupon is accessed by the central processing unit during analysis of the resonance frequency.

6. A method according to claim 1, wherein a graphical display of the calculated quality, density and/or stiffness is produced.

7. A method according to claim 1, wherein the probe is provided with markings along its length to indicate the depth to which it is inserted into the aperture.

8. A method according to claim 1, wherein the probe is provided with a threaded or spiral external profile.

9. A method according to claim 1, wherein the size and profile of the probe is matched to that of a drill bit used to create the aperture.

10. A method according to claim 1, wherein the excitation means for exciting the elongate probe are bonded to the probe.

11. A method according to claim 1, wherein the excitation means comprises a coil acting directly upon the probe.
